(19) United States
(12) Patent Application Publication
(10) Pub. No.: US 2023/0270724 A1
(43) Pub. Date: Aug. 31, 2023

(54) PDE3 inhibitors for treating viral infections

(71) Applicant: Breakthrough Medical Research B.V., Almere (NL)

(72) Inventors: Jan Beute, Almere (NL); Henricus Bartholomeus Benraad, Berg en Dal (NL)

(73) Assignee: Breakthrough Medical Research B.V., Almere (NL)

(21) Appl. No.: 18/005,976

(22) PCT Filed: Jul. 20, 2021
(86) PCT No.: PCT/EP2021/070272

§ 371 Date: Jan. 19, 2023

(30) Foreign Application Priority Data
Jul. 20, 2020 (EP) 20186774.4
Dec. 18, 2020 (EP) 20215550.3

(51) Int. Cl. A61K 31/4174; A61K 31/675; A61P 31/14

(52) U.S. Cl. CPC A61K 31/4174; A61K 31/675; A61P 31/14

(57) ABSTRACT

The invention relates to PDE3-inhibitors for use in the treatment of a viral infection. The PDE-3 inhibitors achieve a rapid symptomatic relief in subjects, with the advantage of maintaining antiviral activity. Because respiratory failure can be prevented by treatment with PDE3-inhibitors, respiratory failure can be averted, no mechanical ventilation is needed, and overall ICU/hospital time is significantly shorter

Early intervention through treatment with phosphodiesterase-3 (PDE-3) inhibitors such as enoximone (Perfan®) can have beneficial effects when treating viral infections, in particular respiratory viruses such as influenza virus, respiratory syncytial virus, parainfluenza viruses, metapneumovirus, rhinovirus, coronaviruses, adenoviruses, and bocaviruses. Respiratory viruses are usually transmitted by direct hand to surface to hand contact and/or aerosol inhalation, and replicate in both upper and lower airways. Cellular and humoral immunity are both activated in response to respiratory viral infections as well as neural pathways, which contribute to distant inflammatory effects. Respiratory viruses are responsible for a wide variety of clinical syndromes including the common cold, acute otitis media, laryngitis, sinusitis, pneumonia, bronchiolitis, influenza, and exacerbations of asthma and chronic obstructive pulmonary disease. Diagnosis of respiratory viral infections is primarily clinical and is further supported by laboratory techniques such as antigen detection, culture, serology and more recently nucleic acid detection. Preventive strategies are based on avoidance of risk factors and vaccination when indicated. Treatment modalities include over-the-counter and non-specific remedies along with a small number of specific antiviral medications. COVID-19 is an example of a virial infection wherein patients present with dyspnoea and impending exhaustion, thereby preventing ventilation.

COVID-19 is a disease caused by a new Coronavirus (SARS-CoV-2) characterized by a severe upper and/or lower respiratory tract infection. Worldwide, the virus has now (June 2020) been officially diagnosed in more than 9 million people and nearly 480,000 people have died from the effects of the virus. Approximately 50,000 infections have been confirmed in the Netherlands and 6,100 people have died from the effects of the virus.

At present, all treatments given for this infection are off-label because no standard care or effective drug is yet available; out of necessity, only the symptoms can be treated. COVID-19 patients regularly end up on ventilation at the ICU. Recently, however, ventilation in COVID-19 patients has been shown to be associated with many complications and high mortality. COVID-19 patients enrolled in the ICU show severe edema of the lung, a decrease in serum albumin, and also appear to have a relatively low heart rate (consistent with SARS and ARDS image). Some patients worsen after 2 weeks of illness due to sterile inflammation. In addition, they have poor gas exchange, following breathlessness symptoms. Not uncommon with and after a COVID-19 infection are bacteria and fungal co-infections as well as cardiological and haematological (thromboembolic) complications.

Different treatment options that can address different aspects of COVID-19 are currently being investigated. Some options target the virus entering the respiratory epithelium through the ACE2 receptor, or against virus replication. Others are agents that interfere with the consequences of the virus infection, via the activated inflammatory routes and/or important inflammatory components. A possible vaccine is a hitherto empty option (www.theguardian.com/world/2020/may/22/why-we-might-not-get-a-coronavirus-vaccine).

Another treatment strategy is early intervention in the chain of reactions caused by SARS-CoV-2, for example, by protecting and enhancing the natural function of the lung cells by means of PDE3 inhibitors (such as enoximone). This can be achieved by making structural cells less sensitive to the harmful effects of COVID-19-induced mediators and cytokines, and inhibiting the production of these cytokines and mediators. This may prevent serious lung damage during COVID-19 infection, as well as the associated deterioration of lung function.

PDE3 inhibitors are known in the art, with referenced to: improvement of the cilia function of the epithelial cells in the respiratory system; decreasing the production of pro-inflammatory cytokines in epithelial cells; having a direct effect on the cell structure and the shape of the endothelial cells such that the cytoskeleton of the endothelial cell is corrected so that vessels no longer leak, which results in the reduction or prevention of pulmonary edema and improvement of gas exchange; supporting the pumping power of the heart; reduction of the extravasation of granulocytes from the bloodstream to the lung tissue, which prevents (sterile) inflammation; and prevention of mast cell degranulation, which is usually associated with mast cell activation known to contribute to COVID-19-induced inflammation.

The PDE3 inhibitor enoximon appears to be as effective against pro-inflammation as methylprednisolone, with the advantage of maintaining antiviral activity, as seen with patients with cardiopulmonary bypass surgery. Enoximon is an existing PDE3 inhibitor, approved and safe drug that can in principle be used quickly. An additional advantage is that enoximon is relatively cheap.

However, uses of this substance known in the art particularly relate to its intravenous administration for treatment of heart failure. EP2581082 discloses the use of enoximone in the treatment of status asthmaticus wherein enoximone is used to relieve bronchoconstriction. Beute et al. disclose use of enoximone in allergic airway inflammation (DOI: 10.1172/jci.insight.94888). WO2015/160249 teaches aroylimidazolones for use in treating allergic conditions.

Further aroylimidazolones are known, for example from Schnedler et al. (J Med Chem. 1986 May; 29(5):860-2) and from BE883856 and U.S. Pat. No. 4,405,635.

There is a need for pharmacological treatment of viral infections. There is a need for small molecules that are useful for treating viral infections. There is a need for treatment of respiratory viruses. There is a need for reducing the impact of respiratory viral infection on patients. There is a need for reducing the amount of fatigue experienced by patients suffering respiratory viral infections. There is a need for reducing the duration of hospital stay for patients suffering respiratory viral infections. There is a need for reducing the duration of intensive care required for patients suffering respiratory viral infections. There is a need for reducing the amount of mechanical ventilation required for patients suffering respiratory viral infections.

### DESCRIPTION OF THE INVENTION

The invention provides a PDE3-inhibitor such as an aroylimidazolone or a pharmaceutically acceptable salt thereof for use in the treatment of a viral infection. Aroylimidazolones, particularly aroyl-2H-imidazol-2-on, or a pharmaceutically acceptable salt thereof, are widely known in the art. An exemplary aroylimidazolone is enoximone or a pharmaceutically acceptable salt thereof. Therefore, a preferred PDE3-inhibitor is milrinone or an aroylimidazolone, and a preferred aroylimidazolone is enoximone.

The invention therefore concerns a method of treating, preventing, ameliorating, or suppressing a viral infection or a symptom of a viral infection, the method comprising administering to a subject in need thereof an effective amount of one or more PDE-3 inhibitors, preferably as defined herein below. The effective amount is preferably an amount as defined herein below.

Alternatively, the invention pertains to a PDE-3 inhibitor, preferably as defined herein below, for use in treating, preventing, ameliorating, or suppressing a viral infection or a symptom of a viral infection, preferably by administration of an effective dose of the compound as defined herein below.

The viral infection is preferably an infection by a respiratory virus, such as RSV, a pneumovirus, an influenza virus or a coronavirus. More preferably, the viral infection is an infection by a coronavirus, such as SARS-CoV or SARS-CoV-1, MERS-CoV or SARS-CoV-2. Most preferably, the viral infection is an infection by SARS-CoV-2, i.e. is COVID-19.

The medical use herein described is formulated as a compound as defined herein for use as a medicament for treatment of the stated condition(s) (e.g. by administration of an effective amount of the compound), but could equally be formulated as i) a method of treatment of the stated condition(s) using a compound as defined herein comprising a step of administering to a subject an effective amount of the compound, ii) a compound as defined herein for use in the manufacture of a medicament to treat the stated condition(s), wherein preferably the compound is to be administered in an effective amount, and iii) use of a compound as defined herein for the treatment of the stated condition(s), preferably by administering an effective amount. Such medical uses are all envisaged by the present invention.

Aroylimidazolone

The invention describes a new use of aroylimidazolone such as aroyl-2H-imidazol-2-on, preferably enoximone or a pharmaceutically acceptable salt thereof. Enoximone is a compound that belongs to the group that can be referred to as aroyl-2H-imidazol-2-on. In particular, the invention involves the use of the compound 1,3-dihydro-4-methyl-5-[4-(methylthio)benzoyl]-2H-imidazol-2-on, also referred to as 4-methyl-5-{[4-methylsulfanyl)phenyl]carbonyl}-2,3-dihydro-1H-imidazol-2-on, or a pharmaceutically acceptable salt thereof. Aroylimidazolones of the invention are preferably 4-aroyl-2H-imidazol-2-ones. Examples of aroyl-2H-imidazol-2-ones are 4-benzoyl-1,3-dihydro-2H-imidazol-2-one; 1,3-dihydro-4-(4-nitrobenzoyl)-2H-imidazol-2-one; 4-benzoyl-1,3-diacetyl-1,3-dihydro-2H-imidazol-2-one; 4-benzoyl-1,3-dihydro-5-(lower alkyl)-2H-imidazol-2-one; 4-benzoyl-1,3-diacetyl-1,3-dihydro-5-methyl-2H-imidazol-2-one; 1,3-dihydro-4-(hydroxybenzoyl)-2H-imidazol-2-one; 1,3-dihydro-4-(hydroxybenzoyl)-5-(lower alkyl)-2H-imidazol-2-one; 1,3-diacetyl-1,3-dihydro-4-(3,4-dimethylbenzoyl)-2H-imidazol-2-one; 1,3-dihydro-4-(3,4-dihydroxybenzoyl)-2H-imidazol-2-one; 1,3-dihydro-4-methyl-5-(4-nitrobenzoyl)-2H-imidazol-2-one, 4-(3-aminobenzoyl)-1,3-dihydro-2H-imidazol-2-one, 4-(4-aminobenzoyl)-1,3-dihydro-2H-imidazol-2-one, and 4-(4-aminobenzoyl)-1,3-dihydro-5-methyl-2H-imidazol-2-one Hereinafter, the terms “enoximone” and “aroyl-2H-imidazol-2-on” will mainly be used, whereby all specific compounds mentioned above are also referred to. A preferred aroylimidazolone is a aroyl-2H-imidazol-2-on or a pharmaceutically acceptable salt thereof, even more preferably it is enoximone or a pharmaceutically acceptable salt thereof.

Treatment

The term “PDE-3 inhibitor” as used herein refers to a substance which is capable of inhibiting or suppressing the (enhanced) expression and/or functional activity of PDE-3 in cells or in subjects but preferably does not have any or significant inhibitory effect on the expression and/or functional activity of other PDE enzymes. PDE-3 and PDE3 are used interchangeably. A PDE3 inhibitor is a drug which inhibits the action of the phosphodiesterase enzyme PDE3. They are generally used for the therapy of acute heart failure and cardiogenic shock. The compounds for use according to the invention are capable of selectively reducing the level of PDE-3, preferably without significantly affecting the levels of other PDEs, such as e.g. PDE1, PDE2, or PDE4, more preferably without reducing the levels of one or more other PDEs, such as PDE1, PDE2, or PDE4, to a level which provides an effect associated with a reduction in said PDE.

In the context of this invention, the activity of an enzyme preferably relates to the amount of reactions it catalyzed per time unit. In the context of this invention, the amount of expression of an enzyme preferably relates to the amount of enzyme molecules that are present at a given moment, or that are produced, preferably in a cell, per time unit.

The term “inflammation” will be understood by those skilled in the art to include any condition characterized by a localized or a systemic protective response, which may be elicited by physical trauma, infection, chronic diseases, such as those mentioned hereinafter, and/or chemical and/or physiological reactions to external stimuli (e.g. as part of an allergic response). Any such response, which may serve to destroy, dilute or sequester both the injurious agent and the injured tissue, may be manifested by symptoms mediated by for instance enhanced mPGES-1 expression or activity, e.g. heat, swelling, pain, redness, dilation of blood vessels and/or increased blood flow.

The term “inflammation” is also understood to include any inflammatory disease, disorder or condition per se, any condition that has an inflammatory component associated with it, and/or any condition characterized by inflammation as a symptom, including, inter alia, acute, chronic, ulcerative, specific, allergic, infection by pathogens, immune reactions due to hypersensitivity, entering foreign bodies, physical injury, and necrotic inflammation, and other forms of inflammation, like cancer inflammation, known to those skilled in the art. The term thus also includes, for the purposes of this invention, inflammatory pain, pain generally and/or fever.

An “effective amount” of a compound is an amount of a compound which, when administered to a subject, is sufficient to reduce or eliminate either one or more symptoms of a disease, or to retard the progression of one or more symptoms of a disease, or to reduce the severity of one or more symptoms of a disease, or to suppress the manifestation of a disease, or to suppress the manifestation of adverse symptoms of a disease. An effective amount can be given in one or more administrations, such as two or more, three or more, four or more, five or more, 6 or more, 7 or more, 8 or more, 9 or more, or 10 or more. Preferably, two administrations per day are provided.

The “effective amount” of that may be combined with the carrier materials to produce a single dosage form will vary depending upon the host to which the active ingredient is administered and the particular mode of administration. The unit dosage chosen is usually fabricated and administered to provide a desired final concentration of the compound in the blood.

The effective amount (i.e. the effective total daily dose), preferably for adults, is herein defined as a total daily dose of about 5 to 2000 mg, or about 10 to 1000 mg, or about 20 to 800 mg, or about 30 to 800 mg or about 30 to 700 mg, or about 20 to 700 mg or about 20 to 600 mg, or about 30 to 600 mg, or about 30 to 500 mg, about 30 to 450 mg or about 30 to 400 mg, or about 30 to 350 mg or about 30 to 300 mg or about 50 to 600 mg, or about 50 to 500 mg, or about 50 to 450 mg, or about 50 to 400 mg or about 50 to 300 mg, or about 50 to 250 mg, or about 100 to 250 mg or about 150 to 250 mg. In the most preferred embodiment, the effective amount is about 100 to 200 mg, most preferably in two doses of 50 mg or two doses of 100 mg.

Alternatively, the effective amount of the compound, preferably for adults, preferably is administered per kg body weight. The total daily dose, preferably for adults, is therefore about 0.05 to about 40 mg/kg, about 0.1 to about 20 mg/kg, about 0.2 mg/kg to about 15 mg/kg, or about 0.3 mg/kg to about 15 mg/kg or about 0.4 mg/kg to about 15 mg/kg or about 0.5 mg/kg to about 14 mg/kg or about 0.3 mg/kg to about 14 mg/kg or about 0.3 mg/kg to about 13 mg/kg or about 0.5 mg/kg to about 13 mg/kg or about 0.5 mg/kg to about 11 mg/kg.

The total daily dose for children is preferably at most 200 mg. More preferably the total daily dose is about 5 to 200 mg, about 10 to 200 mg, about 20 to 200 mg about 30 to 200 mg about 40 to 200 mg, or about 50 to 200 mg. Preferably, the total daily dose for children is about 5 to 150 mg, about 10 to 150 mg, about 20 to 150 mg about 30 to 150 mg about 40 to 150 mg, or about 50 to 150 mg. More preferably, the total daily dose is about 5 to 100 mg, about 10 to 100 mg, about 20 to 100 mg about 30 to 100 mg about 40 to 100 mg, or about 50 to 100 mg. Even more preferably, the total daily dose is about 5 to 75 mg, about 10 to 75 mg, about 20 to 75 mg about 30 to 75 mg about 40 to 75 mg, or about 50 to 75 mg.

Alternative examples of dosages which can be used are an effective amount of the compounds of the invention within the dosage range of about 0.1 μg/kg to about 300 mg/kg, or within about 1.0 μg/kg to about 40 mg/kg body weight, or within about 1.0 μg/kg to about 20 mg/kg body weight, or within about 1.0 μg/kg to about 10 mg/kg body weight, or within about 10.0 μg/kg to about 10 mg/kg body weight, or within about 100 μg/kg to about 10 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight. Other dosages which can be used are about 0.01 mg/kg body weight, about 0.1 mg/kg body weight, about 1 mg/kg body weight, about 10 mg/kg body weight, about 20 mg/kg body weight, about 30 mg/kg body weight, about 40 mg/kg body weight, about 50 mg/kg body weight, about 75 mg/kg body weight, about 100 mg/kg body weight, about 125 mg/kg body weight, about 150 mg/kg body weight, about 175 mg/kg body weight, about 200 mg/kg body weight, about 225 mg/kg body weight, about 250 mg/kg body weight, about 275 mg/kg body weight, or about 300 mg/kg body weight.

Compounds of the present invention may be administered in a single daily dose, or the total daily dosage may be administered in divided dosage of two, three or four times daily.

Compounds of the present invention may be administered in an initial first dose concentration, then a subsequent dose(s) of a different concentration. In a preferred embodiment, compounds of the present invention may be administered in an initial first higher dose concentration, then a subsequent lower dose(s) concentration. In an alternative preferred embodiment, compounds of the present invention may be administered in an initial first lower dose concentration, then a subsequent higher dose(s) concentration.

In a preferred embodiment of the invention, “subject”, “individual”, or “patient” is understood to be an individual organism, preferably a vertebrate, more preferably a mammal, even more preferably a primate and most preferably a human. Other preferred mammals are cats, dogs, frets, bats, and pangolins.

In a further preferred embodiment of the invention, the human is an adult, e.g. a person that is 18 years or older. In addition, it is herein understood that the average weight of an adult person is 62 kg, although the average weight is known to vary between countries. In another embodiment of the invention the average weight of an adult person is therefore between about 50-90 kg. It is herein understood that the effective dose as defined herein is not confined to subjects having an average weight. Preferably, the subject has a BMI (Body Mass Index) between 18.0 to 40.0 kg/m2, and more preferably a BMI between 18.0 to 30.0 kg/m2. Preferred subjects are obese subjects. An obese subject preferably has a BMI over 30 kg/m2, more preferably over 35 kg/m2, even more preferably over 40 kg/m2.

Alternatively, the subject to be treated is a child, e.g. a person that is 17 years or younger. In addition, the subject to be treated may be a person between birth and puberty or between puberty and adulthood. It is herein understood that puberty starts for females at the age of 10-11 years and for males at the age of 11-12 year. Furthermore, the subject to be treated may be a neonate (first 28 days after birth), an infant (0-1 year), a toddler (1-3 years), a preschooler (3-5 years); a school-aged child (5-12 years) or an adolescent (13-18 years). In preferred embodiments the subject is an elderly subject, preferably having an age of at least 40, more preferably at least 50, even more preferably at least 60, still more preferably at least 65, most preferably at least 70. Other preferred subjects have an age of at least 35.

Acute respiratory distress syndrome (ARDS) is a preferred type of pulmonary dysfunction, and a type of respiratory failure associated with fast onset of widespread inflammation in the lungs. Symptoms can include shortness of breath, rapid breathing, and blue skin coloration (central and/or peripheral cyanosis). Survivors often experience a decreased quality of life. Viral infections, particularly respiratory viral infections, may cause ARDS. A preferred treatment of as described herein is a treatment or amelioration or prevention of viral infection-associated ARDS. A subject to be treated in this context is preferably a subject suffering from a viral infection, or suspected of suffering from a viral infection, while not suffering ARDS, or while not suffering severe ARDS. For ARDS in general, the underlying mechanism involves diffuse injury to cells which form the barrier of the microscopic air sacs of the lungs (alveoli), surfactant dysfunction, activation of the immune system, and dysfunction of the body's regulation of blood clotting. In effect, ARDS impairs the lungs' ability to exchange oxygen and carbon dioxide. Diagnosis is based on a PaO₂/FiO₂ ratio (ratio of partial pressure arterial oxygen and fraction of inspired oxygen) of less than 300 mm Hg despite a positive end-expiratory pressure (PEEP) above 5 cm H₂O. The viral infection associated with and/or causing the ARDS preferably is an infection by a respiratory virus, such as RSV, a pneumovirus, an influenza virus or a coronavirus. More preferably, the viral infection is an infection by a coronavirus, such as SARS-CoV or SARS-CoV-1, MERS-CoV or SARS-CoV-2. Most preferably, the viral infection is an infection by SARS-CoV-2, i.e. is COVID-19.

Treatment of viral infection associated pulmonary dysfunction such as ARDS is preferably further combined with mechanical ventilation, optionally together with treatments directed at the underlying cause such as administration of a bradykinin receptor inhibitor. Ventilation strategies include using low volumes and low pressures. If oxygenation remains insufficient, lung recruitment maneuvers and neuromuscular blockers may be used. If this is insufficient, extracorporeal membrane oxygenation (ECMO) can be used. In preferred embodiments the PDE3 inhibitor for use according to the invention ameliorates progression of the viral infection, preferably to obviate ICU admission. Amelioration preferably comprises at least one of an absence of need for sedation, an absence of need for non-invasive pulmonary support or mechanical ventilation, an absence of cytokine release syndrome, and survival. More preferably each of these are comprised.

In preferred embodiments the PDE3 inhibitor for use according to the invention is for use in improving oxygenation saturation (SpO2) in the subject. Preferably, oxygenation saturation is improved by at least 1%, 2%, 3%, 4%, 5%, 6%, 7%, 8%, 9% or 10%. Within these embodiments, a subject to be treated preferably has an oxygenation of below 95%, such as below 90%. Oxygen saturation and the measurement therefore will be known to one skilled in the art, such as using a pulse oximeter.

In the context of the invention, treating, curing, and/or stabilizing the disease or condition associated with viral infection may mean that:
at least a symptom of this disease or condition has been improved,
at least a parameter associated with this disease or condition has been improved.

Depending on the identity of the disease or condition, a symptom may be at least one of the following symptoms:
itching of the skin, red skin and urticarial
sneezing, coughing, nasal blockage, nasal discharge, itching of the nose, itching of the eye and itching and/or inflammation of the oral mucosa
wheezing, chest tightness, dyspnoea

The improvement may be measured and/or visible (i.e. significant improvement) after one hour of the onset of the treatment, or 2, 3, 4, 5, 6, 7, 8, 9, 10, 11, 12, 13, 14, 15, 16, 17, 18, 19, 20, 21, 22, 23, 24 hours, or after one day, two days, four days, or after at least one week, one month, six months after the onset of the treatment or more.

It is encompassed by the invention that the treatment may consist of the administration of a single dose, or two doses or three doses of the compound. It is also encompassed by the invention, that the treatment may comprise or consist of an administration frequency of no more often than once per day, or preferably once every four days, or preferably no more often than six, five, four, three, or two times per week, or more preferably no more often than weekly or less often than once per week or each two weeks or each three weeks or monthly or even less frequently.

During the course of the treatment, the symptom may also occur less and less frequently. As a result a subject may adapt the frequency of administration of enoximone depending on the severity of occurrence of a symptom. Enoximone may be administrated once or twice a week as a first dose and depending on the evolution of the frequency of the occurrence of a symptom, it may subsequently be administrated every week, subsequently every two weeks. A symptom may be assessed by interview or anamnesis, or alternately by known tests or appropriate parameters.

In a preferred embodiment, the present invention provides a new indication or a new medical use for aroyl-2H-imidazol-2-on, preferably for the substance enoximone, which can be used for treating viral infection, particularly COVID-19, with direct as well as long term maintenance results. Enoximone can effectively prevent the need for mechanical ventilation and reduce hospital stay duration. Related to the description above, an embodiment of the invention is the use of aroyl-2H-imidazol-2-on, preferably enoximone, for ameliorating and/or treating a disease or condition in a subject, wherein said disease or condition is COVID-19, preferably fatigue associated with COVID-19.

In a preferred embodiment, the invention provides aroyl-2H-imidazol-2-on, preferably enoximone, for treating ARDS associated with viral infection, particularly ARDS associated with COVID-19. The results are both direct as well as long term. Enoximone can effectively prevent ARDS and thus prevent the need for mechanical ventilation, which reduces hospital stay duration. The aroyl-2H-imidazol-2-on, preferably enoximone, was found to be effective in treating, more particularly preventing, this severe inflammation event.

Form of the Substance

According to the present invention, aroyl-2H-imidazol-2-on, preferably enoximone is a solid substance which can for example be processed into tablets, suppositories, enemas, vaginal tablets, suspensions, powders, (transdermal) patches and creams. In the above forms, the patients can manage the intake or application themselves, obviously in accordance with the prescriptions of the treating physician. An injectable preparation (intravenous, subcutaneous, intracutaneous or intramuscular), for example a depot preparation with delayed release, is another possible form. This can be administered in an outpatient centre or by a GP or physician.

Administration and Quantity

According to the present invention, the active substance aroyl-2H-imidazol-2-on, preferably Enoximone, can be taken both as a fluid and as a solid and can also be applied to the skin. The fluid oral form of administration is a suspension. The possible solid oral forms are dispersible tablets, effervescent tablets, coated tablets (capsules or granules), delayed release tablets or modified release tablets (capsules or granules) (slow release), liquid soft gel capsules, gums or chewing gums, sublingual preparations, capsules, powders, granules, patches, enemas, tablets, preferably vaginal tablets, suppositories or creams or ointment, oral solutions, oral suspensions. Preferred tablet or capsule formulations are provided in the examples. An injectable depot preparation (sub/intracutaneous or intramuscular) is also conceivable. A preparation suitable for intravenous administration is also possible, for example, a preparation suitable for perfusion, intermittent or continual or a combination thereof. The term perfusion as used herein is also known as infusion and includes intravenous therapy, such as through central lines or peripheral lines. In a preferred embodiment, the active substance aroyl-2H-imidazol-2-on, preferably Enoximone, is in the form of a preparation suitable for intravenous administration, for example in a solution comprising saline and/or dextrose.

For the cream version, enoximone can be added to a dermatological medium in a solid or fluid form, possibly containing further excipients. Sublingual preparation is attractive as it exhibits a quick resorption (i.e. less than 60 s) and it is unobstructive. Gum or chewing preparation is attractive especially for children as it is not perceived as a drug or medicament. It is also not obstructive. Enoximone may also be formulated to be administrated as a soap, cream, shower gel/cream and/or shampoo (topical use). This is especially attractive for skin disorder patients. They often are very tender to normal soaps and shampoos.

When enoximone is administered orally, it is preferable that the quantity of active substance is situated in a range from 0.01 to 2 mg per kg body weight, preferably 0.01 to 0.8 mg/kg, preferably 0.01 to 8 mg/kg, preferably at least 0.05 mg/kg, more preferably at least 0.1 mg/kg, even more preferably at least 0.2 mg/kg and most preferably at least 0.25 mg/kg.

Furthermore, the quantity is preferably at most 1.5 mg/kg, more preferably at most 1 mg/kg, even more preferably at most 0.8 mg/kg and most preferably at most 0.5 mg/kg. The maximum daily dose of the active substance is preferably 2 mg/kg of body weight yet at most 250 mg/kg body weight, more preferably at most 200 mg/kg body weight, even more preferably at most 150 mg/kg body weight.

The solid form of administration can for example be made available in dosage units of 5, 10 and 20 mg, relative to the quantity of aroyl-2H-imidazol-2-on, preferably enoximone.

When aroyl-2H-imidazol-2-on, preferably enoximone, is administered in inhalation form, it is preferable that the effective release of the active substance is situated in a range from 0.01 to 15 mg, preferably from 0.01 to 8 mg preferably at least 0.05 mg, more preferably at least 0.1 mg, even more preferably at least 0.2 mg and most preferably at least 0.3 mg, for example 0.5 mg. Furthermore, the quantity is preferably at most 15 mg, more preferably at most 10 mg, even more preferably at most 8 mg and most preferably at most 5 mg.

The substance can be made available as a suspension, for example in 5, 10, 20, 50 or 100 ml containers. The aroyl-2H-imidazol-2-on, preferably enoximone, content can for example be 1 mg/ml. In one embodiment the enoximone is prepare in syringes intended for dilution, for example 10 mg of enoximone in a 4 ml syringe is then diluted to 5 mg/ml with 2 ml NaCl 0.9%.

When aroyl-2H-imidazol-2-on, preferably enoximone is administered in cream form, it is preferable that the effective concentration (w/w) of aroyl-2H-imidazol-2-on, preferably enoximone in the cream is situated in a range from 0.01 to 10%, preferably at least 0.05%, more preferably at least 0.8%, and preferably at most 8%, more preferably at most 5%, even more preferably at most 3%.

Enoximone may also be formulated to be administrated as a soap, cream, shower gel/cream and/or shampoo in dosage forms of 5, 10, 15, 20, 25, 50, or 100 mg per dose.

In an embodiment of the invention, the use of an aroyl-2H-imidazol-2-on, preferably enoximone or a pharmaceutically acceptable salt thereof is its use for the preparation of a medicament for ameliorating and/or treating a disease or condition in a subject, wherein said disease or condition is preferably viral infection such as COVID-19.

In an embodiment of the invention, a pharmaceutical composition comprising aroyl-2H-imidazol-2-on, preferably enoximone or a pharmaceutically acceptable salt thereof in an active effective quantity, preferably in a dosage unit of 5, 10 or 20 mg, based on the quantity of the active ingredient, is provided. In said pharmaceutical composition, said aroyl-2Himidazol-2-on can be enoximone or a pharmaceutically acceptable salt thereof. Each feature of this pharmaceutical composition has been already defined herein.

The dosage and frequency of the above-mentioned forms can be adjusted according to the body weight, the patient's experiences, and findings from future tests and experiments.

Initial dosages can be estimated from in vivo data, e.g., animal models, using techniques that are well known in the art. One having ordinary skill in the art could readily optimize administration to humans based on animal data. The amount of molecules administered will, of course, be dependent on the subject being treated, on the subject's weight, the severity of the affliction, the manner of administration and the judgment of the prescribing physician. The moment of administration can be based on the moment of the occurrence of symptoms, for example according to the patient's experiences with regard to the disorder. The moment of administration can be based on a dosage regime that can comprise daily administrations, or less frequent administrations such as once every four days, twice a week, or weekly or each two weeks or each three week or monthly or even less frequently. A relatively less frequent administration (i. e. once every four days, twice a week, or weekly or each two weeks or each three week or monthly or even less frequently) is attractive since possible toxicity or side effect is minimized and patient's freedom and self-image are optimized.

In addition such less frequent administration allows a de-escalation regime: the patient himself may decrease the frequency of administration based on his own perceived symptoms.

Aroyl-2H-imidazol-2-on, preferably enoximone, can be administered or taken in an appropriate manner in daily, multi-daily, weekly or multi-weekly doses.

Formulations

According to the present invention, aroylimidazolone such as aroyl-2H-imidazol-2-on, preferably enoximone is a solid substance which can for example be processed into tablets, suppositories, enemas, vaginal tablets, suspensions, powders, (transdermal) patches and creams. In the above forms, the patients can manage the intake or application themselves, obviously in accordance with the prescriptions of the treating physician. An injectable preparation (subcutaneous, intracutaneous or intramuscular), for example a depot preparation with delayed release, is another possible form. This can be administered in an outpatient centre or by a GP or physician. Unlike the existing form of administration consisting of a fluid, dissolved through a special procedure (corresponding use in heart failure), which must always be administered in a clinical situation, these forms of application are new and much more accessible.

In an embodiment, aroyl-2H-imidazol-2-on, preferably enoximone is not injected intravenously, more preferably not injected via a bolus injection. The term “bolus” as used herein is also known as an intravenous push. In an alternative embodiment, aroyl-2H-imidazol-2-on, preferably enoximone is injected intravenously, more preferably injected via a bolus injection, infusion or a combination of a bolus injection and infusion. In a preferred embodiment a bolus of 20 mg is followed by 10 mg/h using a perfusor. In a more preferred embodiment, a bolus of 20 mg (0.25 mg/kg) enoximone is followed by 10 mg/h (0.125 mg/kg/hr) using a perfusor (i.e. approximately 2.1 mg/kg/min) for approximately 24 to 48 hours.

In one embodiment the preparation comprises an active drug substance, filler, binder, disintegrant and lubricant. In a further embodiment the preparation is a compression formulation, prepared by dry blending of components, comprising aroyl-2H-imidazol-2-on, preferably enoximone, lactose, microcrystalline cellulose, croscarmellose sodium and magnesium stearate. In a further embodiment the preparation comprises aroyl-2H-imidazol-2-on, preferably enoximone, lactose, povidone, crospovidone and magnesium stearate.

In one embodiment the preparation is a wet granulation tablet, prepared by the mixing of excipients and drug substance with a binder solution using granulation equipment. The addition of extra-granular excipients is also part of the process, therefore, in a further embodiment the wet granulation table comprises aroyl-2H-imidazol-2-on, preferably enoximone, microcrystalline cellulose, povidone, sodium starch glycolate, and magnesium stearate.

In on embodiment, the preparation is a capsule composition comprising, aroyl-2H-imidazol-2-on, preferably enoximone, microcrystalline cellulose, povidone, croscarmellose sodium and hydrophobic silica.

The active drug substance can be in crystalline or amorphous form. Manufactured as direct compression. In one embodiment, the preparation is orodispersible tablets (ODT) comprising an active drug substance, filler, binder, sweetener, glidant, lubricant, and flavours. In a further embodiment, the ODT comprises aroyl-2H-imidazol-2-on, preferably enoximone, mannitol, crospovidone, acesulfame K, hydrophobic silica, magnesium stearate and flavour, such as orange flavour and/or peppermint flavour. Suitable alternatives to the components of the aforementioned solid preparations are known to those in the art.

In one embodiment, the preparation is an aqueous suspension for nasal inhalation wherein the active drug substance is micronized in an aqueous suspension. In a further embodiment the aqueous suspension for nasal inhalation is buffered between pH 3 and pH6, more preferably at pH 4.5. In one embodiment the aqueous suspension for nasal inhalation comprises an active drug substance, viscosity modifier, surfactant, tonicity agent, complexing agent, preservative and carrier. In a further embodiment the aqueous suspension for nasal inhalation comprises aroyl-2H-imidazol-2-on, preferably enoximone, croscarmellose sodium, polysorbate 80, sodium chloride, disodium edetate, benzalkonium chloride and water.

Aroyl-2H-imidazol-2-on, preferably enoximone can be administered or taken in an appropriate manner in daily, multi-daily, weekly or multi-weekly doses.

In an embodiment, aroyl-2H-imidazol-2-on, preferably enoximone is for use as defined herein wherein the subjects show limited or no response to treatment with at least one of the following: beta-2 agonists, parasympatholytics, anticholinergics, aminophyllines, antihistamines, magnesium sulphate, corticosteroids, and cytostatics, preferably with antihistamines.

The inventors have found that enoximone is extremely suitable for the above purposes. Intravenous administration lead to improvements within as little as one minute, and within ten minutes if administered orally. As maintenance medication, it ensures lasting (from a few days to several weeks or several months) improvement of the symptoms.

The inventors have found that treatment of all of the above conditions is surprisingly adequate, and above all, immediate. Surprisingly efficient results were obtained when aroyl-2H-imidazol-2-on, preferably enoximone was administered at low doses and/or at a low frequency. Low doses in this context means 10 mg a day or less than 10 mg per day, or 0.01 to 8 mg per day or less. In preferred embodiments, not more than 10 mg is administered per 3 days. Potential side effects may be circumvented through the use of such low doses. In the context of the invention, low frequency means the present invention allows for dosage regimes that involve an intake schedule featuring intake moments that occur daily, once every four days, weekly, twice a week, preferably six, five, four, or three times a week, more preferably even less often (once per week, once per month, every two, three, four month or even less often), thus relieving the burden on the patient. Treatment with enoximone and the subsequent decrease of complaints and recovery of condition, will improve the quality of life of many patients considerably.

Kit

In one embodiment there is a kit of parts comprising a composition as defined herein, and further comprising an additional pharmaceutical agent that is an antiviral medicament, such as remdesivir.

### Definitions

In this document and in its claims, the verb “to treat” is used preferably to refer to delaying, ameliorating, stabilizing, preventing, or curing a disease or condition, or the progress of a disease or condition, in a subject. More preferably, the disease is ameliorated, stabilized, or cured, more preferably it is ameliorated. Treatment preferably comprises administration of an effective dose of the aroylimidazolone.

A subject to be treated is preferably a subject in need of treatment. Preferably, a subject is a warm-blooded subject, more preferably a mammal. Preferred mammals are humans and domesticated mammals. Preferred domesticated mammals are dogs or horses. In preferred embodiments, a subject is a human or a dog. In preferred embodiments, a subject is a dog. In preferred embodiments, a subject is a human. In preferred embodiments, a subject is not a human.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a product, an assay device respectively a method or a use as defined herein may comprise additional component(s) respectively additional step(s) than the ones specifically identified, said additional component(s), respectively step(s) not altering the unique characteristic of the invention.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

### EXAMPLES

The invention is explained in more detail below with a number of tests and examples, which are not to be construed as limiting the scope of the invention. The invention is not limited to the forms of implementation described in the cases given as examples. The invention also extends to each combination of measures as described above, independently from each other.

### Example 1—Clinical Cases

TABLE 1
Clinical and laboratory characteristics of patients of COVID-19 on the day patient went to ICU for mechanical ventilation

| Subject number | A | B | C | 1 | 2 | 3 | 4 |
|---|---|---|---|---|---|---|---|
| Age,years | 71 | 55 | 63 | 46 | 54 | 70 | 55 |
| Symptoms | Feeling | 7 days | Sick for | 5 days | 7 days | 8-12 may | COPD |
| | sick | worsening | a week. | cough, | of | admission | patient. |
| | with | fever | Tested | fever, | dyspnea | for COPD. | 11 days |
| | fever | (39-40º), | positive | dyspnea | before | 15 may | of |
| | two | headache, | at home. | muscle | admission | SARS- | sickness |
| | weeks | cough. | | pain. | | COV-2 | and |
| | before | | | Anosmia, | | pos. | fever |
| | admission | | | no appetite. | | 19 may | before |
| | | | | | | readmission | admission |
| | | | | | | for | on |
| | | | | | | pneumonia | 5-7 -> |
| | | | | | | | COVID+ |
| | | | | | | | (28-6 neg) |
| Duration of fever, days | 14 | 7 | 7 | 5 | 6 | 12 | 11 |
| Sore throat | − | − | − | − | + | − | − |
| Cough | + | + | − | − | + | + | − |
| Sputum production | + | − | − | − | − | − | − |
| Fatigue | + | + | + | + | + | + | + |
| Shortness of breath | + | + | + | + | + | + | + |
| Nausea or vomiting | − | − | + | − | − | − | − |
| Blood parameters counts | | | | | | | |
| (reference value) | | | | | | | |
| Hemoglobin (6.8-9.3 mmol/L) | 6.9 | 6.8 | 8.1 | 9.0 | 6.5 | 7.5 | 6.2 |
| C-reactive protein (0-10 mg/L) | 116 | 199 | 243 | 6 | 234 | 191 | 173 |
| Albumin (30-50 g/L) | 17 | 19 | 19 | 27 | 22 | 14 | 25 |
| Bilirubin (μmol/L) | 13 | 8 | 18 | | 5 | 8 | 6 |
| D-dimer (0-0.5 mg/L) | 2.1 | 0.21 | 2.8 | 0.54 | 0.33 | 1.3 | 0.47 |
| ESR | | | | | | | |
| Lactate (mmol/L) | 1.5 | 1.1 | 1.2 | 2.9 | 0.8 | 1.3 | 0.7 |
| Procalcitonin (ug/L) | − | − | − | − | − | − | − |
| White blood cell, (4.0-10.0 × 109/L) | 23.7 | 8.5 | 13.8 | 5.3 | 9.7 | 11.0 | 4.4 |
| Neutrophils (2.0-6.0 × 109/L) | 9.0 | 7.6 | 12.5 | 2.8 | 7.9 | 9.7 | 3.6 |
| Lymphocytes (1.0-3.5 × 10⁹/L) | 12 | 0.7 | 0.7 | 1.5 | 1.4 | 0.8 | 0.8 |
| Monocytes (1.0-3.5 × 10⁹/L) | 0.86 | 0.13 | 1.00 | 0.51 | 0.32 | 0.43 | 0.12 |
| Eosinophils (1.0-3.5 × 10⁹/L) | 0.25 | 0.02 | 0.01 | 0.13 | 0.01 | 0.08 | 0.00 |
| Platelet count (1.3-3.6 × 109/L) | 5.30 | 2.00 | 3.11 | 5.12 | 3.16 | 3.85 | 1.52 |

TABLE 2
Duration of hospital stay, chest radiography, mechanical ventilation characteristics and intervention

| Subject number | A | B | C | 1 | 2 | 3 | 4 | p-value |
|---|---|---|---|---|---|---|---|---|
| Age, years | 71 | 55 | 63 | 46 | 54 | 70 | 55 | |
| | | | | | | (COPD | | |
| | | | | | | Gold III) | | |
| Sex (Male/Female) | Male | Female | Male | Male | Female | Female | Female | |
| Date onset disease | 14 Mar. 2020 | 28 Mar. 2020 | 29 Mar. 2020 | 3 May 2020 | 13 May 2020 | 14 May 2020 | 17 Jun. 2020 | |
| Hospital Admission (HA) | 28 Mar. 2020 | 5 Apr. 2020 | 6 Apr. 2020 | 8 May 2020 | 20 May 2020 | 19 May 2020 | 5 Jul. 2020 | |
| | | | | | | (readmission; | | |
| | | | | | | prev. | | |
| | | | | | | admission | | |
| | | | | | | 8-12 May 2020 | | |
| | | | | | | for COPD | | |
| | | | | | | (SARS-CoV-2 | | |
| | | | | | | neg)). | | |
| Duration of disease, days | 14 | 7 | 7 days | 5 days | 7 | 5 | 11 | |
| from onset to admission | | | | | | | | |
| ICU admission [i] | 4 Apr. | 7 Apr. | 8 Apr. | 17 May | 24 May | 24 May | 8 Jul. | |
| | ICU | ICU | ICU | ICU | ICU | ICU | ICU | |
| Days at hospital | 7 | 2 | 2 | 9 | 4 | 5 | 3 | |
| before ICU [ii] | | | | | | | | |
| Days at ICU [iii] | 12 | 13 | 31 | | 5 | 2 days | 5 | <0.05 |
| | | | | | | optiflow | | |
| Days mechanical ventilation | 8 | 10 | 22 | 0 | 0 | 0 | 0 | <0.05 |
| Days at hospital after ICU [iv] | 6 | 7 | 13 | 3 | 4 | — | 7 | <0.05 |
| Date of discharge hospital | 22 Apr. 2020 | 27 Apr. 2020 | 22 May 2020 | 22 May 2020 | 2 Jun. 2020 | —** | 20 Jul. 2020 | |
| Total days in hospital | 25 | 22 | 46 | 14 | 13 | —** | 15 | |
| [=ii + iii + iv] | | | | | | | | |
| Total duration of disease | 39 | 29 | 53 | 19 | 20 | —** | 26 | p ≤ 0.05 |
| (onset-discharge) | | | (to rehab | | | | (to | |
| [=i + ii + iii + iv) | | | facility) | | | | transition | |
| | | | | | | | ward) | |
| CAT-scan: Embolus | enoximone | − | Optiflow | − | − | − | − | − |
| Right lower lobe + | | | started at | | | | | |
| small embolus | | | 55% oxygen | | | | | |
| more peripherally. | | | and | | | | | |
| Centrilobular emphysema. | | | 60 L flow | | | | | |
| Infiltrations and fibrosis | | | | | | | | |
| consistent with COVID-19 | | | | | | | | |
| in both upper lobes. | | | | | | | | |
| Chest X-Ray: Bi-lateral | enoximone | − | Optifow | − | − | − | − | − |
| infiltrations | | | started at | | | | | |
| | | | 100% oxygen | | | | | |
| | | | and | | | | | |
| | | | 60 L flow | | | | | |
| CAT-scan: Disseminated | enoximone | − | Optiflow | − | − | − | − | − |
| infiltrations and spotty | | | started | | | | | |
| consolidations | | | at 100% | | | | | |
| | | | oxygen and | | | | | |
| | | | 50 L flow | | | | | |
| Cat-scan: No embolus. | enoximone | − | Optiflow | − | − | − | − | − |
| Bi-latera lground | | | started | | | | | |
| glass, notably lower | | | at 100% | | | | | |
| lobes, right lower | | | oxygen and | | | | | |
| lobe consolidated | | | 50 L flow | | | | | |
| CAT-scan: | Chloroquine | − | PCV | 26 | 10 | 28.6 | + | + |
| Embolus in left lower | (600-800 | | followed | | | | | |
| lobe and small one right | mg/day) | | by assist | | | | | |
| mediobasal. Infiltrations | | | ventilation | | | | | |
| predominantly in | | | | | | | | |
| upper lobes. | | | | | | | | |
| Spotty infiltrations | | | | | | | | |
| compatible with [symbol] | | | | | | | | |
| CAT-scan: No embolus, | Chloroquine | − | PCV | 24 | 10 | 30.1 | + | + |
| bi-lateral spotty | (600-800 | | followed | | | | | |
| infiltrations, some | mg/day) | | by assist | | | | | |
| emphysema | | | ventilation | | | | | |
| CAT-scan: | Chloroquine | − | PCV | 26 | 14 | 34 | + | + |
| No embolus | (600-800 | | followed | | | | | |
| Bilateral spotty | mg/day) | | by assist | | | | | |
| 16onsole-dations. [symbol] | | | ventilation | | | | | |
| Chest radiography | Intervention | vv-ECMO | Specific | Plateau | PEEP | Compliance | Prone | Neuro- |
| findings* | 1^{st} day | (days) | characteristics | pressure | (cm H2O) | (ml/cmH2O) | position | muscular |
| | of ICU | | of mechanical | (cm H2O) | | | | blockade |
| | admission | | ventilation | | | | | |
| Inhaled pulmonary | − | − | + | − | − | − | − | |
| vasodilators | | | | | | | | |
| Vasopressors | − | + | + | − | − | − | − | |
| Prone position | + | + | + | − | + | − | + | |
| Neuromuscular blockade | Yes | No | Yes | No | No | No | No | |
| Prone position | | | | | | | | |
| Inhaled pulmonary | − | − | + | − | − | − | + | |
| vasodilator | | | | | | | | |
| Vasopressors | − | + | + | − | − | − | + | |

*Diffuse bilateral infiltrates
Abbreviations: vv-ECMO, veno-venous extracorporeal membrane oxygenation. [symbol]
indicates data missing or illegible when filed

### Example 2—Formulations of Aroylimidazolones

Non-limiting examples of formulations are provided. In each of the examples below, each of the specific substances used in combination with enoximone may have been replaced by another equivalent substance having the same function as indicated in the tables (i.e other filler, binder, disintegrant, lubricant, glidant, sweetener, flavor, viscosity modifier, surfactant, tonicity agent, complexing agent, preservative and/or carrier).

### 2.1 Immediate Release (IR) Tablets or Capsules

The active drug substance can be in crystalline or amorphous form.

2.1.1 Direct Compression IR Tablet

Direct compression formulations are prepared by dry blending of components.

| Component | mg | Action |
|---|---|---|
| Enoximone | 10 | Active drug substance |
| Lactose | 50 | Filler |
| Microcrystalline cellulose | 30 | Filler/binder |
| Croscarmellose sodium | 4.5 | Disintegrant |
| Magnesium stearate | 0.5 | Lubricant |
| Total | 100 | |

2.1.2 Direct Compression IR Tablet

| Component | mg | Action |
|---|---|---|
| Enoximone | 10 | Active drug substance |
| Lactose | 75 | Filler |
| Povidone | 10 | Binder |
| Crospovidone | 4.5 | Disintegrant |
| Magnesium stearate | 0.5 | Lubricant |
| Total | 100 | |

2.1.3 Wet Granulation IR Tablet

Wet granulation involves mixing of excipients and drug substance with a binder solution using granulation equipment. Addition of extra-granular excipients is also part of the process; the formula below shows the overall composition without differentiating intra and extragranular components.

| Component | mg | Action |
|---|---|---|
| Enoximone | 10 | Active drug substance |
| Microcrystalline cellulose | 158 | Filler |
| Povidone | 10 | Binder |
| Sodium starch glycolate | 21 | Disintegrant |
| Magnesium stearate | 1 | Lubricant |
| Total | 200 | |

2.1.4 Capsule Composition

Granules manufactured as above and filled into capsules (e.g. hard gelatin).

| Component | mg | Action |
|---|---|---|
| Enoximone | 20 | Active drug substance |
| Microcrystalline cellulose | 108 | Filler |
| Povidone | 6 | Binder |
| Croscarmellose sodium | 15 | Disintegrant |
| Hydrophobic silica | 1 | Glidant |
| Total | 150 | |

2.2 Orodispersible Tablets (ODT)

The active drug substance can be in crystalline or amorphous form. Manufactured as direct compression.

| Component | mg | Action |
|---|---|---|
| Enoximone | 10 | Active drug substance |
| Mannitol | 82.6 | Filler |
| Crospovidone | 5 | Binder |
| Acesulfame K | 0.5 | Sweetener |
| Hydrophobic silica | 0.5 | Glidant |
| Magnesium stearate | 1 | Lubricant |
| Orange flavour | 0.3 | Flavour |
| Peppermint flavour | 0.1 | Flavour |
| Total | 100 | |

2.3 Aqueous Suspension for Nasal Inhalation

The active drug substance is micronised. Aqueous suspension buffered at pH 4.5:

| Component | % (w/w) | Action |
|---|---|---|
| Enoximone | 0.06 | Active drug substance |
| Croscarmellose sodium | 0.15 | Viscosity modifier |
| Polysorbate 80 | 0.05 | Surfactant |
| Sodium chloride | 0.9 | Tonicity agent |
| Disodium edetate | 0.05 | Complexing agent |
| Benzalkonium chloride | 0.02 | Preservative |
| Water for injection | q.s. | Carrier |

It can be concluded based on the tests described above that treatment with aroylimidazolone such as enoximone, possibly in addition to the traditional treatment, is a very adequate and advantageous approach for subjects suffering from viral infection such as COVID-19. Other treatments such as mechanical ventilation can be obviated and total duration of hospital stay can be reduced by several days. In addition to the fact that quality of life can be improved considerably for a subject thanks to treatment with aroylimidazolones, this treatment offers substantial cost savings to society and can mitigate the unhinging effect of viral infections.

### Example 3

Research into the mechanism of allergic diseases shows that the a similar pathophysiology also occurs with COVID-19 infections. The inventors found that Enoximon treats many of the symptoms associated with allergic disease, with the added benefit of leaving normal anti-viral (and anti-fungal) defenses intact.

Dosage, Side Effects and Practical Application

The initial indication of enoximone was heart failure; doses were used up to >2000 mg per day. A much lower dose, 50 mg orally 3 times a day, has been described in the Essential Trials of Metra (n=1854) and was found to be similar in placebo to adverse events in patients with advanced heart failure. For the COVID-19 indication presented here, we used an initial bolus of 20 mg (0.25 mg/kg) enoximone IV, followed by 10 mg/h (0.125 mg/kg/hr) using a perfusor (i.e., approximately 2.1 mcg/kg/min) for approximately 24 to 48 hours; this dosage is based on our experience with off-label treated asthmatics (250 patient years) who received 25 mg orally no more than once daily. In England, enoximone is used orally at a similar dose in children with pulmonary hypertension at home without hemodynamic control. Since the COVID image shows a significantly higher mortality than COPD and asthma, we dosed in such a way that underdosing is avoided.

No side effects are expected at the dosage suggested here; monitoring for side effects must of course take place. Enoximone in this dosage could be used in addition to other agents; no contraindications are known. The application was off-label.

The usual concentration of enoximone to be administered in Dutch hospitals is 2.5 mg/ml for intravenous use; we propose to prepare syringes of 10 mg=4 ml, this by diluting 2 ml Perfan® (5 mg/ml) with 2 ml NaCl 0.9%. Thus, 10 doses of 10 mg can be prepared from one ampoule of Perfan®.

In Rivierenland Hospital, Tiel, based on existing literature and our findings with the use of PDE3 inhibitor enoximon in asthma and status asthmatic, we treated 4 patients with PDE3 inhibitor enoximon (Perfan®, Carinopharm GmbH, Germany) and thus artificial respiration at the ICU. All four patients were adults (>18 years old) with confirmed SARS-CoV-2 by polymerase chain reaction (PCR).

Upon ICU admission, all patients presented with progressive dyspnoea, deteriorating dyspnoea with an imminent need for mechanical ventilation, a high oxygen need (all had a non-rebreathing mask), indicative of severely impaired alveolar gas exchange, and an imminent respiratory failure. They also showed monosyllabic conversation, use of auxiliary respiratory muscles, and exhaustion due to increased respiratory difficulties caused by decreased pulmonary compliance, implying oedema as a result of capillary leakage probably induced by (pro)inflammatory cytokines.

Statistical Analysis

For statistical analysis regarding mechanical ventilation requirement, we used the Chi-Square tests, Fisher's exact test, suitable for binary data in unpaired samples, i.e. the 2×2 table (SPSS 2.5). Mann-Whitney U test (one-tailed) was used for group comparison of a continuous endpoint, the number of days in the hospital and the number of days on mechanical ventilation (GraphPad Prism 5.0).

Cases

Patient 1—a 46-year-old man with complaints starting 03-05-2020; recorded on 08-05-2020. Transferred to ICU on 1705-2020 due to severe shortness of breath, imminent exhaustion and need for ventilation. Upon arrival at the ICU, the patient was connected to the Optiflow. After explaining and obtaining informed consent, 20 mg enoximone IV was administered, followed by a perfusion of enoximone, which administered 10 mg/h enoximone. Upon entry, patient was monosyllabic; 10 minutes after enoximon was broken in, he was breathing calmly and was able to call his wife. The enoximone was reduced to 0 within 24 hours and the day after, the patient was able to return to the COVID department. Patient was discharged on 22-05-2020.

Patient 2—a 54-year-old woman with complaints from 13-05-2020; recorded on 20-05-2020. Transferred to ICU on 24-05-2020 due to increasing dyspnoea and imminent exhaustion and need for ventilation. On arrival ICU, an attempt was first made to get the patient in better condition with Optiflow alone; after peer consultation, it was decided to propose patient treatment with enoximone. After explanation and informed consent, the patient was given 20 mg of enoximone IV, followed by a perfusion of enoximone, at 10 mg/h. Due to sputum retention, it took a little longer (1 day) before the patient had lost the feeling of dyspnea; she has also been prone on the ICU a number of times (under her own power). After about 2 days, the patient had calmer breathing and could be mobilised.

On 25-05-2020, the enoximon was reduced to 5 mg/h. Enoximone was discontinued on 27-05-2020. The patient returned to the COVID department on 29-05-2020 and the patient was allowed to go home on 02-06-2020.

Patient 3—a 70-year-old female, PCR positive since 5-14-2020. Husband was already SARS-CoV-2 positive. The patient was familiar with COPD Gold III. On 24-05-2020 she was admitted to ICU because of severe shortness of breath (auxiliary breathing muscles in use, shoulders lifted, talking with great difficulty). On arrival to ICU Optiflow was tried, with little result. After explanation and informed consent, the patient received 20 mg enoximon IV, followed by 10 mg enoximon/h by perfuson. After a day and a half, the patient breathed optimally (COPD taken into account) and the Optiflow could be reduced to 0. The patient was able to calmly tell her life story.

Patient 4—a 55 year-old female with respiratory symptoms since 24/06/2020; on 28/06/2020 she was tested negative for SARS-CoV-2. This early test was done because of her prior diagnosis with COPD GOLD II. Her condition deteriorated and she was referred to hospital on 05/07/2020; this time she tested SARS-CoV-2-positive. On 08/07/2020 she was transferred to the ICU (with non-rebreathing mask) because of severe dyspnoea and exhaustion. Enoximone (Additional data: Rationale) was started as above-mentioned and followed by i.v. perfusor (NB: 5 mg/hour during 24 hours). Within 10 minutes patient was able to breath calmly and to communicate normally with the nursing staff. On 13/07/2020 she was back on the COVID ward; on 20/07/2020 she could be discharged.

In summary, following successful reversal of the COVID-19 related symptoms and signs in all four patients, none of the enoximone-treated patients required mechanical ventilation and could be safely transferred to the COVID ward for further recovery and subsequent discharge. The enoximone-treated patients could generally be discharged from the hospital within 5-12 days after start of enoximone treatment, while in the three following control subjects this took 18-44 days following start of mechanical ventilation.

Control Group

Four matching prior COVID-19 patients were screened for comparability. One patient was decided not to be include due to a large central pulmonary embolism. Patients were treated in the usual manner, i.e. treatment with Optiflow and intubation with no/insufficient result.

Patient A—a 71-year-old male, admitted on 28-03-2020, in the COVID ward; was moved due to exhaustion on 04-04-2020 to the ICU—intubated, ventilated and directly in the prone position. In total the patient was 12 days ICU, of which 8 days ventilated and 2 periods with prone position. Returned to the COVID department on 16-04-2020 and returned home on 22-04-2020.

Patient B—a 55-year-old woman, admitted on 05-04-2020 in the COVID department; on 07-04-2020 was admitted to ICU with intubation and ventilation. From 09-04-2020 to 12-04-2020 prone position. Prone position again from 13-04-2020 to 15-04-2020. On 17-04-2020 detubation, after which 2 more days Optiflow. Returned to the COVID department on 20-04-2020 and was dismissed on 27-04-2020. A total of 13 days ICU, of which 10 days ventilation with 2 periods prone and 2 days Optiflow.

Patient C—a 63-year-old man, admitted to the COVID ward on 04-04-2020; on 08-04-2020 because of imminent exhaustion went to the ICU, intubated, ventilated and immediately placed in the prone position. In total 31 days ICU, of which 22 days ventilated, in which 3 periods with prone position. Furthermore, period of transient renal insufficiency, without renal replacement therapy. Also a period of atrial fibrillation. Patient has also been treated for pulmonary embolism. On 22-05-2020 patient was discharged to a rehabilitation institute. Enoximone achieved a rapid symptomatic relief in two out of four patients, with full recovery within a few hours (patients 1 and 4), while the other two patients (patients 2 and 3, with comorbid obesity or COPD GOLD III) required a longer time to respond (24-36 hours) due to sputum retention. Compared to controls, the enoximone-treated patients had no need for mechanical ventilation (Chi-Square Tests, Fisher's exact test p=0.029), had a shorter stay in ICU (2-5 days for enoximone-treated patients versus 12-31 days for controls), and an overall shorter stay in hospital (13-15 days for enoximone-treated patients versus 22-46 days for controls) as well as a shorter recovery time (Table 2) (Mann-Whitney U test p=0.05). Blood gas analysis showed a minimal improvement in three out of four patients between before and 1 h after enoximone. The apparent alkalosis is mainly due to hyperventilation (in turn due to patient anxiety). pO₂ levels are all at the right side of the sigmoid curve of saturation, reflecting maximal oxygen saturation despite severe dyspnoea.

The pre-treatment oxygen demand and the key respiratory characteristics upon ICU admission are shown in Table 3. Following enoximone treatment, the oxygenation substantially improved (Table 3—FiO2% decreases as oxygen saturation remains the same or improves; the lower the percentage, the better the oxygenation) and a decrease of edema was seen, as less intercellular leakage was observed, most likely caused by the regeneration of tight junctions. Patient-reported outcome confirmed the observed improvements; all patients were very relieved about their perceived sudden recovery and not needing mechanical ventilation. At the doses used, enoximone was well-tolerated in all patients and no clinically relevant adverse events were observed. In all patients the beneficial effect persisted even after discontinuation of enoximone. Given the nature of this report, which is not based on a formal study protocol, there are no data available on the after-COVID-19-disease wear-off following hospital discharge (e.g. blood gas analysis, imaging, lung function testing/diffusion capacity test, etc.).

TABLE 3
Respiratory characteristics before Enoximone (admission ICU) - after 24 hrs - after 48 hrs.

| | | RESP. | | RESP. | TIDAL | MINUTE | PEAK/PEEP | RESP. | |
|---|---|---|---|---|---|---|---|---|---|
| PATIENT | DATE | SUPPORT | FiO₂ | RATE | VOLUME | VOLUME | PRESSURE | VALUES | TIME |
| A | 4 Apr. 2020 | PCV | 80% | 22 | 475 | 5.1 | 22/14 | — | 18.24 h |
| | 5 Apr. 2020 | PCV | 40% | 20 | 413 | 4.9 | 31/09 | — | 18.24 h |
| | 6 Apr. 2020 | Pr Supp | 40% | 21 | 484 | 6.1 | 14/10 | — | 18.24 h |
| B | 7 Apr. 2020 | PCV | 55% | 20 | 422 | 6.2 | 23/14 | — | 18.18 h |
| | 8 Apr. 2020 | Pr Supp | 40% | 21 | 492 | 10.0 | 16/08 | — | 18.18 h |
| | 9 Apr. 2020 | Pr Supp | 55% | 15 | 433 | 7.6 | 15/10 | — | 18.18 h |
| C | 8 Apr. 2020 | PCV | 55% | 20 | 458 | 6.6 | 23/14 | — | 12.35 h |
| | 9 Apr. 2020 | PCV | 50% | 24 | 435 | 6.4 | 25/10 | — | 12.35 h |
| | 10 Apr. 2020 | PCV | 41% | 24 | 430 | 6.0 | 25/11 | — | 12.35 h |
| 1 | 17 May 2020 | Optiflow | 45% | 16 | — | — | — | 45%/60 L | 16.24 h |
| | 18 May 2020 | none | — | 15 | — | — | — | 6 L O₂ | 16.24 h |
| | | | | | | | | (nasal probe) | |
| | 19 May 2020 | none | — | 16 | — | — | — | 2 L O₂ | 16.24 h |
| | | | | | | | | (nasal probe) | |
| 2 | 24 May 2020 | Optiflow | 65% | — | — | — | — | 65%/50 L | 11.26 h |
| | 25 May 2020 | Optiflow | 60% | — | — | — | — | 60%/60 L | 11.26 h |
| | 26 May 2020 | none | — | — | — | — | — | 6 L O₂ | 11.26 h |
| | | | | | | | | (nasal probe) | |
| 3 | 24 May 2020 | Optiflow | 100% | 24 | — | — | — | 100%/60 L | 22.27 h |
| | 25 May 2020 | Optiflow | 55% | 12 | — | — | — | 55%/50 L | 22.27 h |
| | 26 May 2020 | Optiflow | 60% | 13 | — | — | — | 60%/60 L | 22.27 h |
| 4 | 8 Jul. 2020 | Optiflow | 55% | — | — | — | — | 55%/60 L | 12.51 h |
| | 9 Jul. 2020 | Optiflow | 50% | — | — | — | — | 50%/55 L | 12.51 h |
| | 10 Jul. 2020 | Optiflow | 50% | — | — | — | — | 50%/50 L | 12.51 h |

### CONCLUSION

It is evident that the infection of the enoximone-treated patients was significantly less severe than that of the non-enoximone-treated patients. The length of time on the ICU is significantly shorter, no ventilation was necessary and healing was faster. The consensus is ventilating and waiting to see what happens. Since there is no effective medication, it is also difficult to judge whether steroids, β-mimetics and anti-histamines are actually effective. Enoximon is effective, and shows effects that go beyond what could be expected of mere symptomatic relief. Early intervention seems crucial, at least before the patient derails; prevention or shortening of ventilation is of utmost importance. This not only prevents secondary damage to the patient, but also reduces the occupancy factor of ICUs and reduces the workload for nursing staff. The dosage suggested here costs about € 75.00 per patient per day and, it seems, it only needs to be administered for a few days.

In conclusion, this case series demonstrates that the progressive course of a SARS-CoV-2 infection in COVID-19 patients can be modulated by early treatment with enoximone. Respiratory failure could be averted, no mechanical ventilation was needed and overall ICU/hospital time was significantly shorter.

1. A method of treatment of a viral infection, the method comprising administering aPDE3-inhibitor or a pharmaceutically acceptable salt to a subject in need thereof.

2. The method according to claim 1, wherein the PDE3-inhibitor is enoximone.

3. The method according to claim 2, wherein the viral infection is a coronaviral infection, preferably COVID-19.

4. The method according to claim 3, wherein treatment of a viral infection comprises preventing, ameliorating, suppressing, or curing a symptom or syndrome associated with the viral infection, and wherein the symptom or syndrome is acute respiratory distress syndrome (ARDS) or COVID-19 associated ARDS.

5. The method according to claim 1, wherein the viral infection is a respiratory viral infection such as an infection by human respiratory syncytial virus.

6. The method according to claim 1, wherein treatment of a viral infection comprises preventing, ameliorating, suppressing, or curing a symptom or syndrome associated with the viral infection.

7. The method according to claim 6, wherein the symptom is at least one of:
i) positive test result for SARS-CoV-2 (COVID-19);
ii) pulmonary edema, preferably pulmonary edema that is resistant to corticosteroids, to adrenaline, or to both, preferably to both;
iii) angioedema outside of the lungs, preferably in the gut or of the skin;
iv) unilateral or bilateral ground-glass opacities or clear consolidations as visible on CT scans of the subject, with or without pulmonary fibrosis or symptoms of pulmonary fibrosis;
v) antibodies against Sars-CoV-2 spike antigen;
vi) inflammation, preferably inflammation in the lungs and/or airways;
vii) an age of at least 35, more preferably at least 50, even more preferably at least 60, still more preferably at least 65, most preferably at least 70;
viii) fever;
ix) dry cough;
x) dyspnea;
xi) tachypnea;
xii) increased D-dimers, with or without evidence of thromboembolic events and/or without evidence of vascular leakage, preferably without evidence of thromboembolic events and/or without evidence of vascular leakage;
xiii) having suffered viral infection such as COVID-19 for at least nine days;
xiv) elevated IL-6, elevated TNFα, and/or elevated IL-33;
xv) decrease in serum albumin;
xvi) poor gas exchange;
xvii) organ function impairment, preferably renal, hepatic, and/or myocardial impairment;
xviii) de novo perniosis (chill blains);
xix) de novo experienced cutis marmorata or livedoid patterned rash;
xx) de novo lupus erythematosus
xxi) elevated CRP;
xxii) elevated ferritin;
xxiii) any of xiii-xxii without elevated procalcitonin, preferably all of xiii-xv without elevated procalcitonin;
xxiv) cytokine release syndrome;
xxv) thrombosis;
xxvi) drowning, near drowning, or sensation of drowning in interstitial or alveolar fluid;
xxvii) embolism, preferably pulmonary embolism;
xxviii) vascular leakage, preferably pulmonary vascular leakage;
xxix) emphysema, preferably centrilobular emphysema;
xxx) exhaustion.

8. The method according to claim 1, wherein treatment of a viral infection comprises preventing, ameliorating, suppressing, or curing exhaustion, preferably COVID-19-associated exhaustion.

9. The method according to claim 1, wherein treatment of a viral infection comprises preventing the need for mechanical ventilation of the subject suffering the viral infection,
or wherein treatment of a viral infection comprises reducing the need for intensive care treatment to at most five days.

10. The method according to claim 1, wherein the PDE3-inhibitor is not for achieving bronchodilation or for treating an allergic reaction.

11. The method according to claim 1, wherein the PDE3-inhibitor is administrated at least once per day, preferably at least twice per day, even more preferably three times per day.

12. A composition comprising a PDE3-inhibitor for the method of claim 1.

13. The composition according to claim 12, wherein the composition is formulated for administration in any one of the following forms:
i) in oral form, preferably of a quantity of active substance situated in a range from 0.01 to 2 mg/kg of body weight;
ii) in inhalation form, preferably in a quantity whereby the effective release of active substance is situated in a range from 0.01 to 15 mg;
iii) in topical form, preferably in cream form, whereby the effective concentration (w/w) of PDE3-inhibitor is preferably situated in a range from 0.01 to 10%;
iv) in solid form, preferably as dispersible tablet, effervescent tablet, coated tablet, tablet with delayed release, sublingual preparation, gum or chewing gum, capsule or powder, with an optionally added adjuvant or carrier, most preferably as an effervescent tablet;
v) in fluid form, wherein a PDE3-inhibitor is added to a compatible dissolving, suspending or emulsifying medium;
vi) in cream form, wherein a PDE3-inhibitor is added to a compatible dermatological medium;
vii) as a transdermal patch, preferably in a maximum effective PDE3-inhibitor dose of 100 mg/day, more preferably as a transdermal patch with delayed release;
viii) as an injectable preparation, preferably subcutaneous or intramuscular, more preferably as a depot preparation;
ix) in the form of a sublingual preparation, preferably in dosage forms of 5, 10, 15, 20, 25, 50, or 100 mg per dose;
x) in the form of a gum or chewing gum, preferably in dosage forms of 5, 10, 15, 20, 25, 50, or 100 mg per dose;
xi) in the form of a soap, cream, shower gel, or shampoo, preferably in dosage forms of 5, 10, 15, 20, 25, 50, or 100 mg per dose;
xii) as an intravenously injectable preparation.

14. Kit of parts comprising a composition as defined in claim 12, and further comprising an additional pharmaceutical agent that is an antiviral medicament, such as remdesivir.

15. The method of claim 1, wherein the PDE3-inhibitor aroylimidazolone such as anis.
